(19) United States
(12) Patent Application Publication
(10) Pub. No.: US 2001/0000324 A1
(43) Pub. Date: Apr. 19, 2001

(54) Promotion of cell differentiation by initially passaged cells

(76) Inventors: Ivan T. Todorov, San Diego, CA (US); Kelly I. Scheyhing, Carlsbad, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/726,696

(22) Filed: Nov. 29, 2000

Related U.S. Application Data
(63) Continuation of application No. PCT/US99/11949, filed on May 28, 1999.
(60) Provisional application No. 60/135,114, filed on May 29, 1998.

(51) Int. Cl. C12N 5/00; A61K 35/39

(52) U.S. Cl. 424/93.7; 435/377

(57) ABSTRACT

A method of inducing differentiation of less differentiated passaged cells by contacting or co-cultivating the cells with initially passaged (P0), more differentiated cells of the same cell type. The method produces differentiated cell aggregates, such as pancreatic islet cell aggregates, which are useful for transplantation.

### FIELD OF THE INVENTION

1. The present invention relates to promotion of cell differentiation. More specifically, the invention relates to the ability of initially passaged (P0) differentiated cells to induce the differentiation of expanded passaged cells.

### BACKGROUND OF THE INVENTION

2. Cell differentiation is a process which occurs in the blood, tissues and organs in which a cell population develops a specialized form, character or function differing from that or surrounding cell types. For example, marrow stromal cells are undifferentiated precursor cells which become bone-forming cells called osteoblasts, plasma cells become functional B-cells after antigen selection and islet cell precursors become functional islet cells which secrete insulin in response to glucose challenge. Cells isolated from particular tissues are fully differentiated in that they serve a specialized function.

3. Millions of Americans have Type I (insulin-dependent) diabetes, in which the pancreas has lost its ability to secrete insulin due to autoimmune destruction of the insulin-secreting pancreatic beta cells. Although insulin injections can compensate for the absence of insulin, blood sugar levels can still fluctuate significantly. The elevated blood glucose levels lead to side reactions in which toxic products are formed, leading to serious complications including blindness, kidney disease, circulatory problems, nerve damage, and, ultimately, coma and death.

4. Researchers have tried administering smaller, more frequent doses of insulin and mechanical pumps which mimic the action of the pancreas, but the results have not been satisfactory. Pancreatic transplant is another option; however, the availability of donor pancreases is very limited. In addition, this requires major surgery and is fraught with complications.

5. The most promising option thus far is islet cell transplantation using tissue derived from either cadavers or human fetuses. Although this procedure has been moderately successful, it is difficult to obtain a sufficient number of cells for transplantation into humans.

6. U.S. Pat. No. 5,510,263 describes the expansion of fetal pig pancreatic islet-like cell clusters (ICCs) cultured in contact with an extracellular matrix produced by 804G or NBT-II rat bladder carcinoma cells. U.S. Pat. No. 5,681,587 discloses the successful passaging of adult pig and human islet cells in contact with the same extracellular matrices. U.S. Pat. No. 5,672,361 discloses the growth of islet cells on various non-rat extracellular matrix proteins, referred to as laminin 5. International Publication No. PCT WO 97/16536 discloses co-culturing of freshly isolated, non-proliferated pancreatic islet cells with islet cells which have undergone proliferation. This resulted in cells having a longer viability, stability and insulin secretory activity than did either component itself.

7. There is a constant need for methods of producing large numbers of differentiated cells of various types for transplantation. The present invention addresses this need.

### SUMMARY OF THE INVENTION

8. One embodiment of the present invention is a method of inducing differentiation of cells having a passage number of one or greater, comprising contacting the cells with an effective differentiation-inducing amount of initially passaged (P0) cells of the same cell type to form reaggregated cells. Advantageously, the cells are pancreatic islet cells. Alternatively, the cells are fibroblasts, epithelial cells, endothelial cells, osteoblasts, chondrocytes, hepatocytes, myoblasts or nerve cells. Preferably, the effective amount of P0 cells is between about 1% and 20%. More preferably, the effective amount of P0 cells is between about 5% and about 10%.

9. The present invention also provides reaggregated cells produced by the method described above.

10. Another embodiment of the invention is a method for treating diabetes in a mammal in need thereof, comprising the step of administering to the mammal an effective insulin producing amount of the reaggregated islet cells described above. Preferably, the mammal is a human. In one aspect of this preferred embodiment, the administering step is by implantation under the kidney capsule or direct injection into the liver or peritoneal cavity. Preferably, the cells are placed in an immunoprotective barrier prior to the administering step.

11. The present invention also provides the use of the reaggregated islet cells described above in the preparation of a medicament for treatment of diabetes.

### BRIEF DESCRIPTION OF THE DRAWINGS

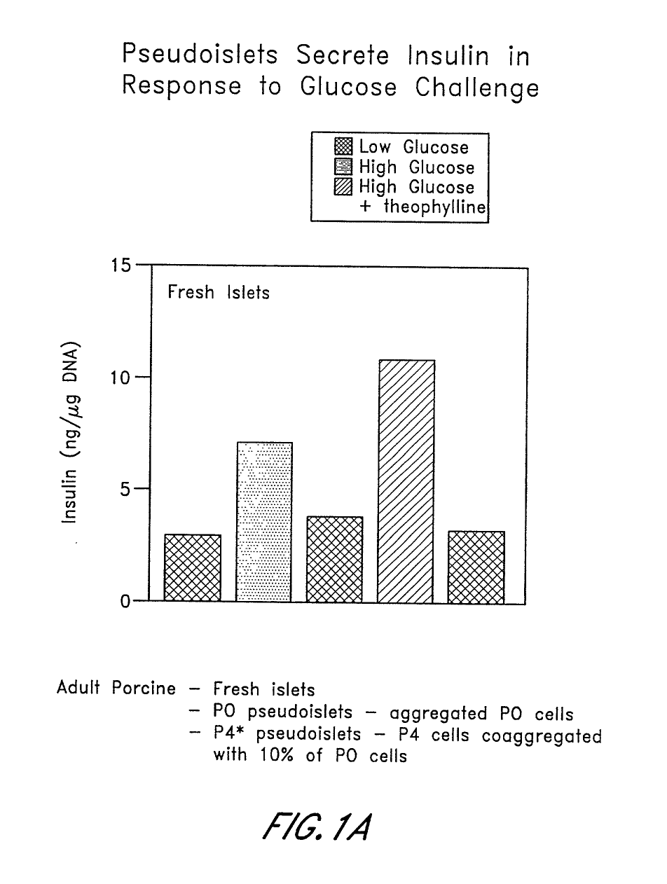

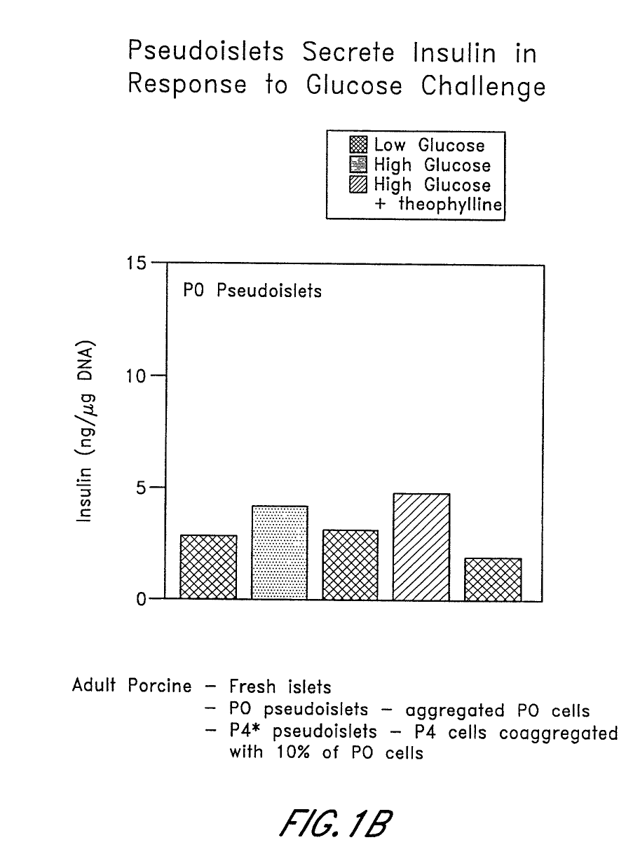

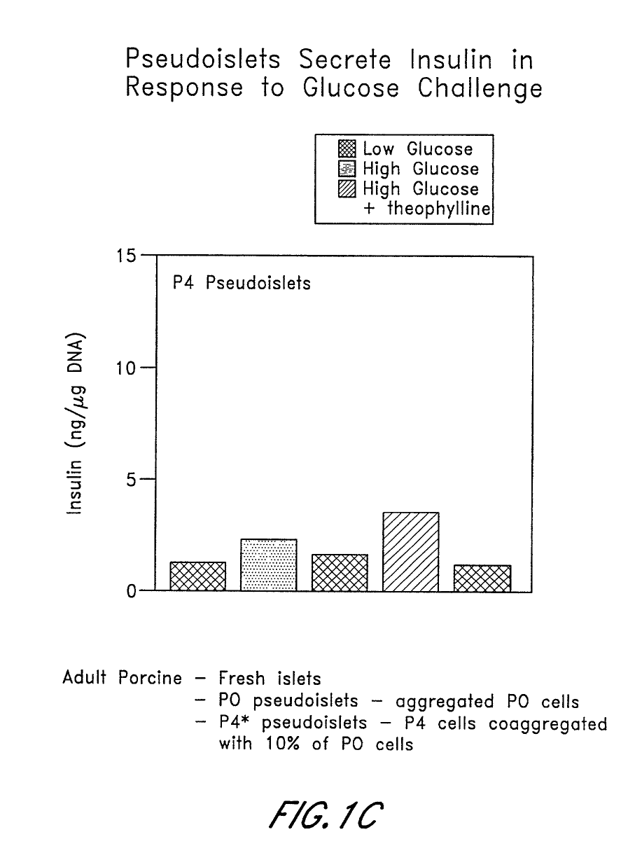

12. FIGS. 1A-1C are graphs showing the glucose responsiveness of expanded porcine fresh islets (FIG. 1A), P0 pseudoislets (FIG. 1B) and P4* islets obtained by co-aggregation of 90% P4 cells with 10% P0 cells (FIG. 1C). The reaggregated “pseudoislets” respond to changes in glucose concentration by secreting insulin in a manner similar to that of freshly isolated islets.

### DETAILED DESCRIPTION OF THE PREFERRED EMBODIMENTS

13. The present invention includes the observation that passaged pancreatic islet cells (≧P1) can be induced to differentiate into clusters called “pseudoislets” in vitro by incubation with a small amount of initially expanded (P0) islet cells. These aggregates are stable for over 10 days in culture, are similar in size and shape to P0 cells and, most importantly, secrete insulin in response to glucose challenge.

14. P0 cells are defined as cells isolated from a tissue or organ which have been initially cultured and expanded in vitro, but have not been passaged in culture. Typically, these P0 cells exhibit an expansion (increase in cell number) of about 10-fold. The term “passaging” indicates that the P0 cells have been initially expanded, removed from their tissue culture dish or flask with trypsin, and reseeded into another dish or flask. The trypsinized, replated cells are referred to as P1 cells. This procedure is then repeated to obtain P2, P3, . . . PX cells. However, when cells are passaged in culture, they gradually lose their specialized functions and become less differentiated (dedifferentiated). For example, when freshly isolated, fully differentiated pancreatic islet cells are passaged in culture, they gradually lose their ability to secrete insulin in response to a glucose challenge.

15. Due to the batch-to-batch variability associated with the use of freshly isolated cells, there is a significant advantage to the use of initially expanded P0 cells for inducing differentiation of cells passaged to P1 or later passages. Because P0 cells have already undergone some dedifferentiation, they afford a greater level of experimental control than do freshly isolated cells.

16. In a preferred embodiment, the amount of initially expanded P0 cells added to expanded cells is between about 1% and 20%. In a more preferred embodiment, the amount of P0 cells is between about 5% and about 10%. During passaging, these cells acquire markers called cytokeratins 7 and 20, markers for ductal cell types which are considered to represent the proliferation compartment in the adult pancreas. The passaged cells gradually lose expression of islet cell markers including insulin, glucagon, somatostatin and GLUT-2, suggesting that they become less differentiated.

17. During the co-aggregation, the P0 cells promote restoration of endocrine function in the resulting “pseudoislets.” Cells within these “pseudoislets” express insulin as assessed by immunofluorescence microscopy and enzyme linked immunosorbent assay (ELISA). In addition, they respond to changes in glucose levels by secreting insulin in a manner similar to freshly isolated islet cells.

18. Although the data presented below relate to stimulating differentiation of expanded isolated cells by P0 cells, promoting differentiation of any cell type which has undergone one or more passages in culture by addition of the corresponding P0 cells is also within the scope of the invention. Such cell types include, but are not limited to, fibroblasts, epithelial cells, endothelial cells, osteoblasts, chondrocytes, hepatocytes, myoblasts and nerve cells. The amount of corresponding P0 cells required for differentiation a particular passaged cell type can be determined by one of ordinary skill in the art by routine experimentation.

19. The subject method is used to produce differentiated cells ex vivo which can then be implanted into a mammal in vivo. For example, pancreatic islet cells can be isolated, passaged and induced to reaggregate and differentiate by the method described above, then implanted into a diabetic mammal, preferably a human. Similarly, hepatocytes can be isolated, expanded and induced to reaggregate ex vivo by addition of initially passaged (P0) hepatocytes. The reaggregated hepatocytes can be implanted into an individual having a liver disorder. Either autologous or heterologous human islet cells can be used to obtain reaggregated differentiated cells for human transplantation. Alternatively, nonhuman, preferably porcine, islet cells can be used. If heterologous human or nonhuman cells are used, it is desirable to place the cells in an immunoprotective barrier prior to transplantation thereof due to potential rejection by the host immune system.

20. Porcine islet cells were cultured and expanded as described in the following example.

### EXAMPLE 1

### Expansion of Porcine Islet Cells

21. Pancreatic islet cells were isolated from Yucatan minipigs (Kenmochi et al., Transplant Proc., 26:3424, 1994). Islets were expanded using conventional tissue culture techniques on flasks coated with purified laminin 5 as described in U.S. Pat. No. 5,510,263 in low serum medium. The purification of laminin 5 is described in U.S. Pat. No. 5,760,179. Cells were passaged with standard trypsinization. Expansion of the islet cell population was evaluated by fluorimetric measurement of intracellular DNA and cell counting as described in U.S. Pat. No. 5,681,587.

22. Expanded cells were characterized by immunofluorescence microscopy. Cells were grown on laminin 5-coated coverslips, fixed with methanol/acetone and processed for immunofluorescence microscopy with antibodies specific for insulin, glucagon and somatostatin. Cells were counterstained with 4,6-diamino-2-phenylindole (DAPI) to visualize DNA/nuclei and to facilitate cell count. For immunofluorescence staining, “pseudoislets” were attached to poly-L-lysine-coated coverslips.

23. Intracellular or secreted insulin was measured by a standard enzyme immunoassay (e.g., insulin enzyme immunoassay kit from Peninsula Laboratories). For measurement of static glucose response, reaggregated “pseudoislets” were subjected to sequential treatment with low (3 mM) glucose, high (16.5 mM) glucose, low glucose, high glucose containing 10 mM theophylline, and finally low glucose. The glucose response is summarized in FIGS. 1A-1C which indicate that the “pseudoislets” respond to changes in glucose concentration by secreting insulin in a manner similar to that of freshly isolated islets.

24. The fold-expansion and insulin content of the fresh islets, P0, P4 and P7 cells are summarized in Table 1, both in cell monolayers and aggregates.

TABLE 1

| | | Monolayers | Aggregates |
|---|---|---|---|
| | Fold expansion | Ins (ng/μg DNA) | Ins (ng/μg DNA) |
| Fresh islets | | | 50.98 ± 9.98 |
| P0 | 8 | 0.902 ± 0.334 | 7.08 ± 4.67 |
| P4 | 600 | 0 | 0 |
| P4* | | | 5.51 ± 0.76* |
| P7 | 10⁵ | 0 | 0 |
| P7* | | | 2.46 ± 0.18* |

*cells co-aggregated with 10% P0 cells

### EXAMPLE 2

### Promotion of Differentiation of Passaged Cells by P0 Cells

25. P4 and P7 islet cells were incubated with initially passaged (P0) cells in a ratio of 9:1. and insulin content was determined. Unexpectedly, the P0 cells promoted differentiation of the P4 and P7 cells as shown by the ability of the reaggregated cells to produce insulin (Table 1). In contrast, the P7 cells alone produced no insulin. The vast majority of the insulin produced was due to the P7 cells, not the P0 cells. As shown in Table 1, P0 islet cell aggregates produced 7.08 ng insulin. Thus, the combination of 90% P7 cells, which produce no insulin, with 10% P0 cells, which would be expected to produce only 0.708 ng insulin, would be expected to produce only 0.708 ng insulin. However, the combination unexpectedly yielded 2.46 ng insulin, over four times the expected amount.

26. It has also been demonstrated that P0 cells can induce differentiation of P3, P5, P6 and P8 cells which regain the ability to secrete insulin in response to glucose challenge. It is contemplated that P0 cells can induce differentiation of any passaged cell population in a less differentiated state (≧P1).

27. The “pseudoislet” aggregates produced by the method of the present invention are transplanted into a diabetic mammal, preferably a human. The aggregated islet cells are implanted under the kidney capsule or injected directly into the liver. The cells are preferably placed in an immunoprotective barrier, such as a permselective membrane, prior to implantation to prevent destruction by the immune system of the mammal into which they are implanted.

### EXAMPLE 3

### Transplantation of Reaggregated Islet Cells Into Diabetic Patients

28. Human diabetes patients are administered between about 10⁵ and 10⁶ islet cells prepared in accordance with Example 2, either by implantation under the kidney capsule or by direct injection into the liver. In addition, transplantation in other ectopic organ locations is also contemplated. Blood glucose levels are monitored over several months and are significantly lower than prior to cellular implantation.

29. The above detailed description of the invention is set forth solely to assist in understanding the invention. It is to be understood that variations of the invention, including all equivalents now known or later developed, are to be considered as falling within the scope of the invention, which is limited only by the following claims.

What is claimed is:

1. A method for inducing differentiation of pancreatic islet cells having a passage number of one (P1) or greater, comprising contacting said cells with an effective differentiation-inducing amount of islet cells which have been initially cultured and expanded in vitro, but have not been passaged in culture, to form reaggregated cells.

2. The method of claim 1, wherein the effective amount of said islet cells which have been initially cultured and expanded in vitro, but have not been passaged in culture, is between about 1% and 20% of the total number of cells.

3. The method of claim 1, wherein the effective amount of said islet cells which have been initially cultured and expanded in vitro, but have not been passaged in culture, is between about 5% and 10% of the total number of cells.

4. Reaggregated cells produced by the method of claim 1.

5. A method for treating diabetes in a mammal in need thereof, comprising the step of administering to said mammal an effective insulin producing amount of the reaggregated islet cells of claim 1.

6. The method of claim 5, wherein said mammal is a human.

7. The method of claim 5, wherein said administering step is by implantation under the kidney capsule or direct injection into the liver.

8. The method of claim 5, wherein said cells are placed in an immunoprotective barrier prior to said administering step.
